(19) United States
(12) Patent Application Publication
(10) Pub. No.: US 2014/0200485 A1
(43) Pub. Date: Jul. 17, 2014

(54) Umbilical cord blood collection device and method

(71) Applicant: Michael Schaffer, El Paso, TX (US)

(72) Inventor: Michael Schaffer, El Paso, TX (US)

(21) Appl. No.: 13/694,878

(22) Filed: Jan. 14, 2013

(51) Int. Cl. A61B 5/15; A61M 1/00

(52) U.S. Cl. CPC A61B 5/150038; A61M 1/008
USPC 600/576; 604/317

(57) ABSTRACT

An apparatus and method for collecting blood from the umbilical cord of a newborn into a standard vacuum collection tube is disclosed wherein the apparatus comprises a funnel shaped body with a plurality of stabilizing supports, an upper inlet and a lower outlet, a cylindrical receiving receptacle attached to one side of the body, a channel connecting the lower outlet to the cylindrical receiving receptacle, and a needle with flow passage contained therein attached to that end of the channel in contact with the cylindrical receiving receptacle.

### DOMESTIC PRIORITY

This application claims the benefit of the filing date for Provisional Application No. 61/5870645, filed Jan. 16, 2012.

### BACKGROUND OF THE INVENTION

1. Field of the Invention

This invention relates to an umbilical cord collection device and method of using same.

2. Description of the Prior Art

Anyone who has visited a hospital surgical room in recent times, knows very well the painstaking precautions taken to prevent unwanted exposure to patient bodily fluids including blood. Notwithstanding the inherent dangers, it is necessary for a patient's bodily fluid to be sampled. Umbilical cord blood must be sampled to ascertain blood type as well as Rh factor. It may even be necessary to preserve an infant's umbilical cord blood for the stem cells such blood contains.

Historically, umbilical cord blood was collected in one of two ways. First, a hypodermic needle could be inserted into the umbilical cord to withdraw the necessary blood. The blood would then be transferred to a rubber stoppered collection tube. Second, the free end of the umbilical cord, that not attached to the placenta, is held, over the opening of a test tube and the cord blood allowed to drip or drain into such test tube. The problem with both these types of procedures were that it could not eliminate the hazard that operating/deliver room personnel might be exposed to the cord blood which might be contaminated with all sorts of infectious diseases.

In an attempt to provide a safer and more efficient way to collect umbilical cord blood, Michael D. Grossman, et al, in U.S. Pat. No. 5,915,384, issued Jun. 29, 1999, for Medical Body Fluid Sampler Device, and in U.S. Pat. No. 5,342,328, issued Aug. 30, 1994, disclosed a funnel shaped apparatus which allowed cord blood to collect and gravity drain into a collection tube. Anyone that has had to gravity flow any type of fluid, let alone fluid that may contain body cells and tissue, knows the problems inherent in this approach, i.e., the flow becomes restricted. Once flow is restricted, most likely the sample of blood will be lost and/or hospital personnel exposed to the blood when attempts are made to remove the restriction.

Peter Yong in U.S. Patent Publication No. 2006/0039833, published Feb. 23, 2006, and U.S. Patent Publication No. 2007/0025886, published Feb. 1, 2007, for Biological Specimen Collection, Transportation, and Dispensing System, in FIG. 81 discloses an apparatus for the collection of umbilical cord blood. The primary emphasis of the Yong disclosure was for use in urine collection. In order to collect urine, i.e., allow a user to urinate into the collection device, one would need a collection device that was relatively tall and narrow as is shown in Yong. In lay person's vernacular, one would need a cup. It is not necessary that the Yong cup be stable when placed on a table because that is not when the sample is being collected. As seen in FIG. 81 of Yong, the needle used to withdraw the collected umbilical cord blood is long running almost the entire height of the cup. The tube, identified as item 762 in Yong, into which the cord blood is drawn, would no doubt increase the instability of the Yong cup, shifting the center of gravity of the cup far to the left. One can clearly see in FIG. 81 of Yong that the cup would surely fall over in its depicted embodiment. In Yong however, being primarily used to collect urine, the stability of the device was not an issue.

Charles David Cawood, in two US Patents, U.S. Pat. No. 4,557,274 issued Dec. 10, 1985, for Midstream Urine Collection and U.S. Pat. No. 5,766;136 issued Jun. 16, 1998 for Midstream Urine Collector with Deflection Shield, discloses a device which arguably could be used for umbilical cord blood collection. Like Yong, since the primary emphasis is for urine collection, the device in Cawood must be tall and narrow. As can be seen in FIG. 6 of U.S. Pat. No. 4,557,274, once urine is collected, tube 29 is forced down onto needle 27. This requires force and without the legs shown in FIG. 6, the collection apparatus would surely tip over. The legs shown, however, cannot overcome the inherent instability of the Cawood apparatus.

### SUMMARY OF THE INVENTION

It is, therefore, an object of the present invention to provide a simple, efficient and stable way to collect cord blood while reducing the potential for exposure to cord blood. Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is an illustration of blood collection device.

FIG. 2 is an illustration of the blood collection device showing the collection tube inserted.

FIG. 3 is a side view illustration of blood collection device

FIG. 4 is an plan view illustration of the blood collection.

FIG. 5 is an illustration of blood collection device depicting a splash cap.

FIG. 6 is an illustration of the blood collection device depicting a removable portion and a base portion.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

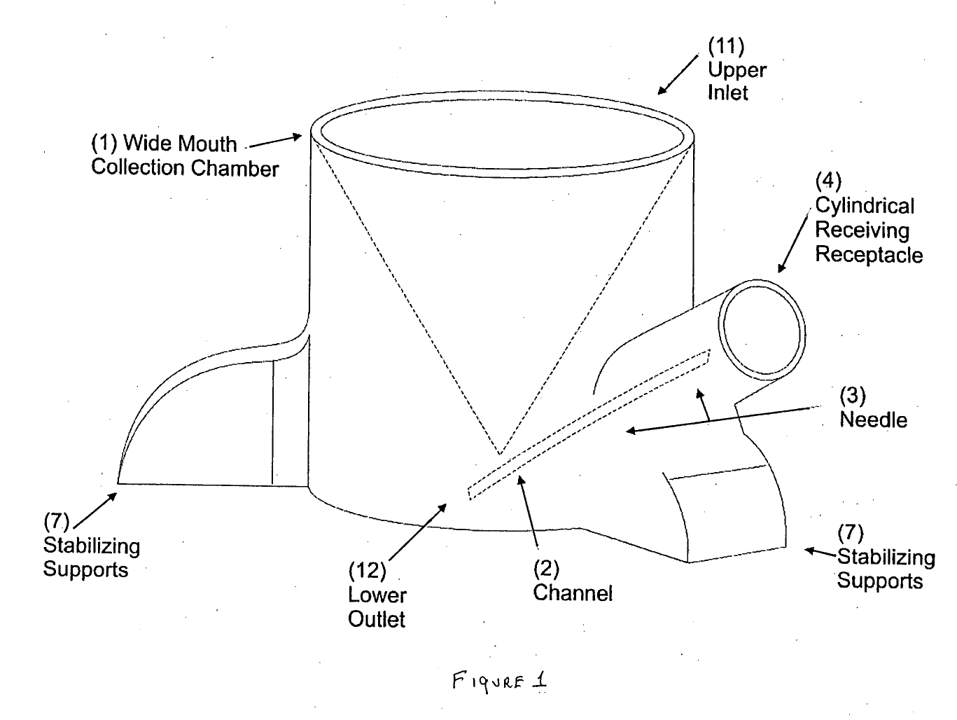

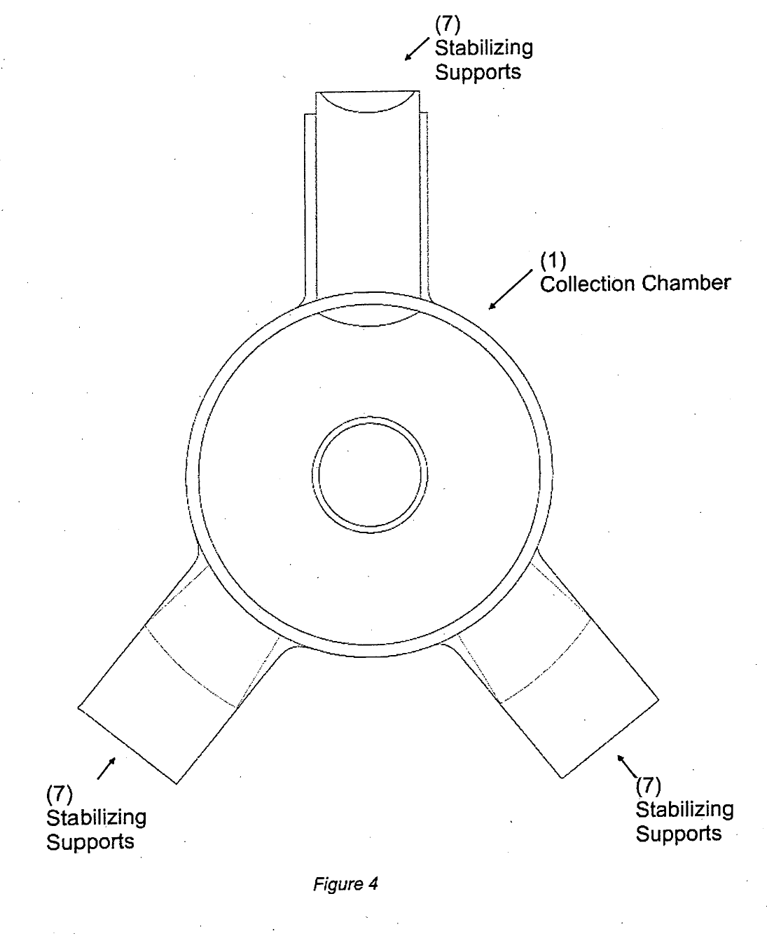

As can be seen in FIG. 1, the umbilical cord blood collection apparatus of the present invention consists of a body with funnel shaped interior surfaces (1) and having an upper inlet (11) and lower outlet (12). To provide stability for said body (1) and ease of blood collection, the body is in general, twice as wide as it is high. While any number of body (1) supports (7) can be utilized, as shown in FIG. 1 and FIG. 4, the present invention uses three such stabilizing supports (7).

While any modern day method of construction would could be used to manufacture the apparatus of the present invention, the use of injection molding results in a lightweight, one piece body (1) with no means required to attach the cylindrical receiving receptacle (4) to said body. In other types of manufacture, the cylindrical receiving receptacle (4) can be attached to the body using many different types of means commonly known in the field such as glue.

At the bottom of the funnel shaped interior surfaces of the body (1) is the lower outlet (12). Umbilical cord blood is allowed to drain into the upper inlet and flow down and out the lower outlet (12). Blood exits the lower outlet (12) through the channel (2). At the end of the channel is a passageway containing needle (3). The needle (3) can be attached to the channel (2) using any conventional method such as glue.

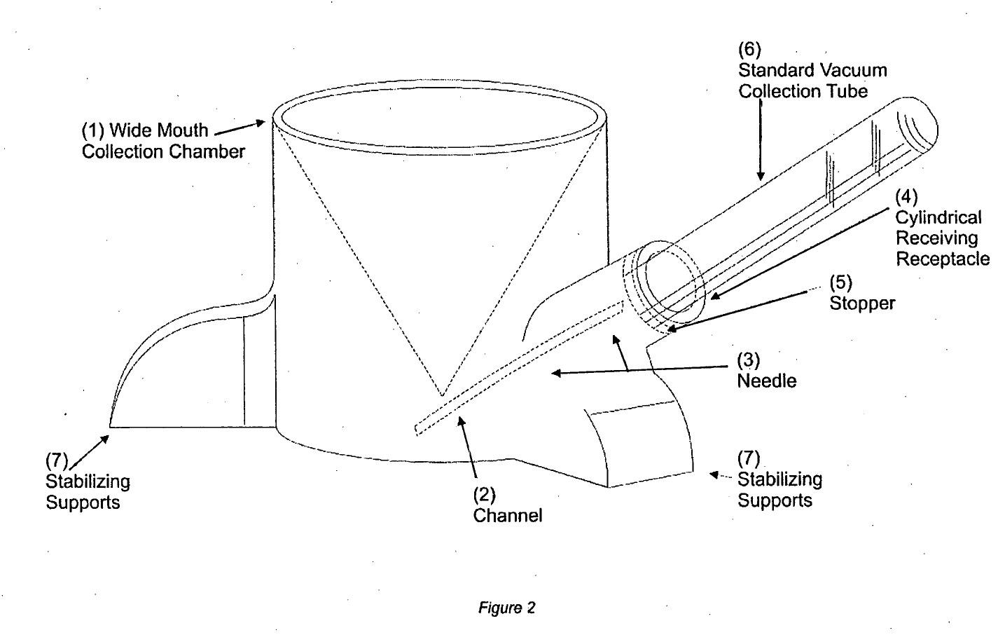

Shown in FIG. 2 is a standard vacuum collection tube (6) inserted into the cylindrical receiving receptacle (4). At the end of the collection tube (6) first inserted into the cylindrical receiving receptacle is a stopper (5). Standard vacuum collection tubes do not contain air. Once the collection tube is force downward into the cylindrical receiving receptacle (4), the stopper (5) is punctured, allowing blood to flow into the collection tube (6). The stopper (5) is self sealing therefore, when the collection tube (6) is removed from the cylindrical receiving receptacle (4), the blood is trapped within the collection tube (6).

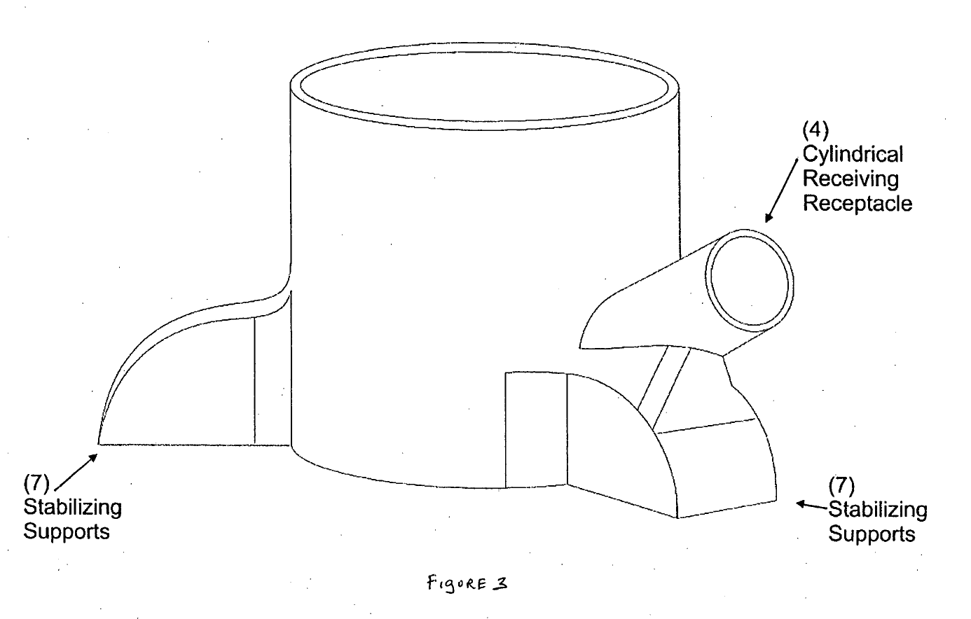

The cylindrical receiving receptacle (4) is shown in FIGS. 1-6, angled away from the body (1). This angle is necessary to allow leverage when forcing the collection tube (6) onto the needle while still permitting good blood flow. An angle greater than 0 degrees and less than 90 degrees, in the range of 25 to 60 degrees, preferably 45 degrees has been seen to work best.

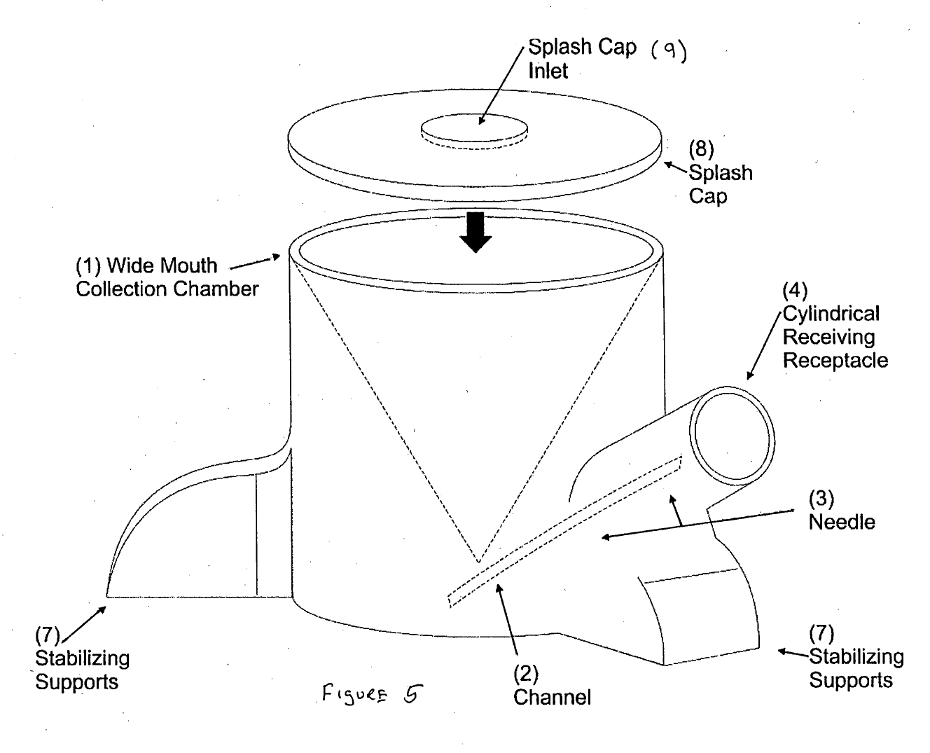

Blood from an umbilical cord generally does not flow fast, however, it can and it can also splatter. For this reason, in another embodiment of the present invention shown in FIG. 5 is a splash cap (8) with splash cap inlet (9). The umbilical cord can be placed into to the splash cap inlet (9) allowing blood into the body (1) or blood can be allowed to simply drain through the inlet (9).

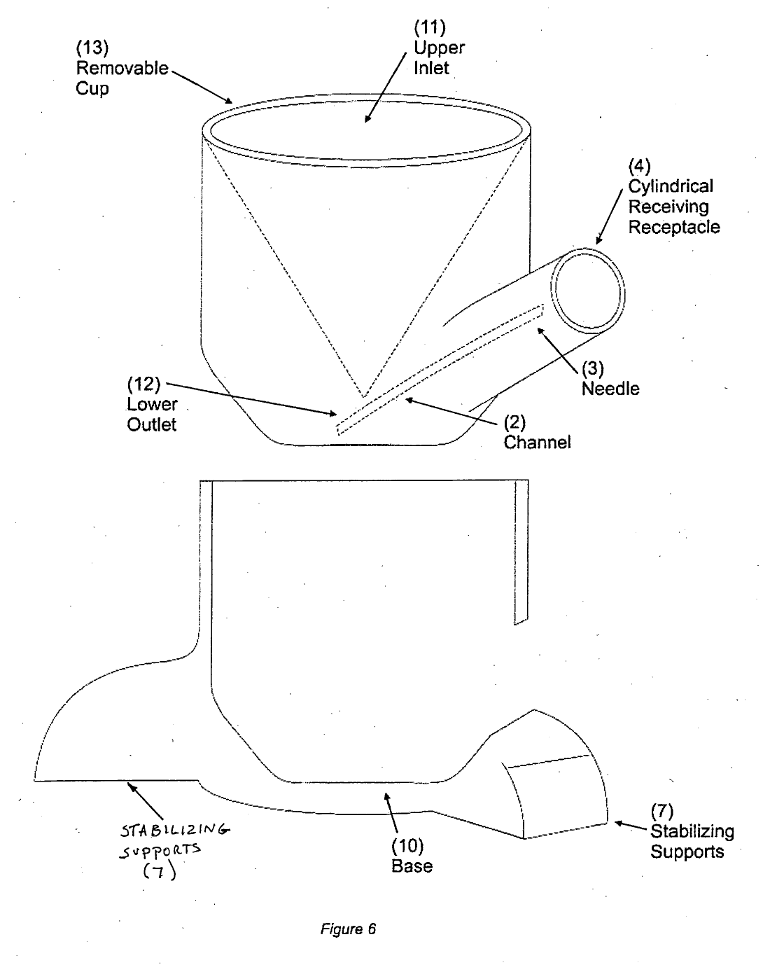

In still another embodiment of the present invention, as shown in FIG. 6, the body can be made of two pieces, a base (1) and a removable cup (13). The removable cup (13) contains the upper inlet (11), lower outlet (12), channel (2), needle (3), and cylindrical receiving receptacle (4). The base (10) is shaped slightly larger than the removable cup (13) so as to allow placement of the removable cup (13) within the base (10). The base (10) utilizes the stabilizing supports (7). In this manner, the removable cup (13) is disposed after each use while the base (1) can be re-used.

While the preferred embodiments have been shown and described, it will be understood that there is no intent to limit the invention by such disclosure, but rather, is intended to cover all modifications and alternate constructions falling within the spirit and scope of the invention as defined in the appended claims.

1. An apparatus for collecting umbilical cord blood comprising a body with funnel shaped interior surfaces and plurality of stabilizing supports, an upper inlet and a lower outlet, a cylindrical receiving receptacle attached to one side of the body, a channel connecting the lower outlet to the cylindrical receiving receptacle, and a needle with flow passage contained therein attached to that end of the channel in contact with the cylindrical receiving receptacle.

2. The apparatus of claim 1 wherein the number of stabilizing supports is 3.

3. The apparatus of claim 1 wherein the diameter of the body is at least twice the height dimension.

3. The apparatus of claim 1 wherein the angle of attachment between the cylindrical receiving receptacle and body is greater than 0 degrees and less than 90 degrees.

4. The apparatus of claim 1 wherein the angle of attachment between the cylindrical receiving receptacle and body is greater than 25 degrees and less than 60 degrees.

5. An apparatus for collecting umbilical cord blood comprising a body with funnel shaped interior surfaces and having a plurality of stabilizing supports, a removable splash cap with splash cap inlet centrally located thereon, an upper inlet and a lower outlet, a cylindrical receiving receptacle attached to one side of the body, a channel connecting the lower outlet to the cylindrical receiving receptacle, and a needle with flow passage contained therein attached to that end of the channel in contact with the cylindrical receiving receptacle.

6. The apparatus of claim 5 wherein the number of stabilizing supports is 3.

7. The apparatus of claim 5 wherein the diameter of the body is at least twice the height dimension.

8. The apparatus of claim 5 wherein the angle of attachment between the cylindrical receiving receptacle and body is greater than 0 degrees and less than 90 degrees.

9. The apparatus of claim 5 wherein the angle of attachment between the cylindrical receiving receptacle and body is greater than 25 degrees and less than 60 degrees.

10. An apparatus for collecting umbilical cord blood comprising a first body having a plurality of stabilizing supports and a cylindrical receiving receptacle opening along one side of said support; a second funnel shaped body of size and dimension slightly less than that of the first body, an upper inlet and a lower outlet, a cylindrical receiving receptacle attached to one side of said second body, a channel connecting the lower outlet to the cylindrical receiving receptacle, and a needle with flow passage contained therein attached to that end of the channel in contact with the cylindrical receiving receptacle.

11. The apparatus of claim 10 wherein the number of stabilizing supports is 3.

12. The apparatus of claim 10 wherein the diameter of said first body is at least twice the height dimension.

13. The apparatus of claim 10 wherein the angle of attachment between the cylindrical receiving receptacle and said second body is greater than 0 degrees and less than 90 degrees.

14. The apparatus of claim 10 wherein the angle of attachment between the cylindrical receiving receptacle and said second body is greater than 25 degrees and less than 60 degrees.

15. The apparatus of claim 10 further comprising a removable splash cap with splash cap inlet centrally located thereon.

16. A method for collecting a sample of umbilical cord blood comprising the steps of:
holding a section of said cord over the upper inlet of an apparatus for collecting umbilical cord blood comprising a body with funnel shaped interior surfaces and having a plurality of stabilizing supports, an upper inlet and a lower outlet, a cylindrical receiving receptacle attached to one side of the body, a channel connecting the lower outlet to the cylindrical receiving receptacle, and a needle with flow passage contained therein attached to that end of the channel in contact with the cylindrical receiving receptacle;
allowing blood to drip from said cord into the upper inlet of said apparatus;
permitting blood to flow through the lower outlet into the channel;
urging a conventional air-evacuated collection tube downward into the cylindrical receiving receptacle until the stopper membrane of said collection tube is pierced by the needle permitting blood to flow into the collection tube;
removing the collection tube from the cylindrical receiving receptacle.
